(19) United States
(12) Patent Application Publication
(10) Pub. No.: US 2023/0233595 A1
(43) Pub. Date: Jul. 27, 2023

(54) USE OF STAT4 INHIBITORS FOR PREVENTION AND TREATMENT OF ALZHEIMER'S DISEASE

(71) Applicant: New York Medical College, Valhalla, NY (US)

(72) Inventors: Jerry L. NADLER, Briarcliff Manor, NY (US); Patric K. Stanton, Mahopac, NY (US)

(21) Appl. No.: 18/157,305

(22) Filed: Jan. 20, 2023

Related U.S. Application Data
(60) Provisional application No. 63/302,591, filed on Jan. 25, 2022.

(51) Int. Cl. A61K 31/713; A61P 25/28; C07K 16/18; A61K 45/06

(52) U.S. Cl. CPC A61K 31/713; A61P 25/28; C07K 16/18; A61K 45/06

(57) ABSTRACT

The present disclosure provides methods of treatment of multiple neurodegenerative diseases, such as Alzheimer's disease, Parkinson's disease, Huntington's disease, Dementia with Lewy Bodies, Frontotemporal dementia, Chronic Traumatic Encephalopathy, Diabetes, Cerebrovascular disease and Stroke based on the administration of STAT4 inhibitors.

This application claims benefit and priority to U.S. Provisional Application No. 63/302,591, filed Jan. 25, 2022 which is incorporated herein by reference in its entirety.

This invention was made with government support under 3R01 HL 142129-03 awarded by the National Institutes of Health. The government has certain rights in the invention.

### TECHNICAL FIELD

The present disclosure provides methods of treating, delaying the onset, alleviating a symptom, and/or deterring the progression of a neurodegenerative disorder, such as Alzheimer's disease, based on the administration of Signal Transducer and Activator of Transcription 4 inhibitors (herein referred to as “STAT4 inhibitors”).

### BACKGROUND

Alzheimer's disease (AD) is a neurodegenerative disorder that affects more than 40 million people worldwide and it is predicted to exponentially increase in the coming decades. At this time no curative treatment exists underscoring the importance of research on the pathophysiology of the disease, as well as the development of new drugs.

Aging and Alzheimer's disease both exhibit declines in the magnitude of long-term, activity-dependent synaptic potentiation in humans and animals that correlate with declines in cognitive function and long-term memory in a range of behavioral tasks. (STAT4) is a transcription factor that is essential for mediating inflammatory responses to IL12 in lymphocytes and regulating the differentiation of T helper cells. Recent studies support a role for STAT4 signaling in a range of inflammatory diseases, including cardiovascular inflammation associated with diabetes, systemic lupus erythematosus and rheumatoid arthritis. However, it remains unknown what role STAT4 plays in central nervous system diseases, such as Alzheimer's disease.

### SUMMARY

The presently disclosed subject matter provides a method of treating, delaying the onset, alleviating a symptom, and/or deterring the progression of Alzheimer's disease. The method includes administering safely to a subject in need thereof a therapeutically effective amount of a STAT4 inhibitor. The administration of such STAT4 inhibitors is intended to reduce STAT4 mediated inflammation within the nervous system which, as demonstrated herein, has been found to be associated with development of Alzheimer's Disease. Targeted cells within the nervous system include, but are not limited to neurons, multiple glial cell types, vascular epithelial cells of the nervous system and those cells that contribute to inflammation within the nervous system. While the disclosure below is directed to treatment of Alzheimer's disease, the disclosure applies equally as well to treating, delaying the onset, alleviating a symptom, and/or deterring the progression of other neurodegenerative diseases that exhibit chronic, progressive neurological deficits, including but not limited to diabetes, Parkinson's Disease, Huntington's Disease, Lewy Body Disease, and Chronic Traumatic Encephalopathy, Cerebrovascular Disease, Stroke, Frontotemporal Dementia and Traumatic Epilepsy. Indeed, any neurological disease that involves central nervous system inflammation may be treated by drugs that target the STAT4 transcription factor activated inflammatory pathway.

The presently disclosed subject matter provides a method for preventing, or alleviating, the symptoms associated with Alzheimer's disease through administration of a STAT4 inhibitor in a therapeutically effective amount to a subject suspected of suffering from, or at risk of developing Alzheimer's disease. Such subjects may be identified as those having the symptoms of aberrant behavioral changes or cognitive dysfunction associated with Alzheimer's disease and/or pre-Alzheimer's disease

In an embodiment, the subject to be treated may be one suffering from disorders that render a subject more prone for development of neurological disorder such as Alzheimer's. Such disorders include, but are not limited to diabetes, obesity, cardiovascular disorders such as atherosclerosis, to name a few.

Pharmaceutical compositions and formulations for use in treatment of Alzheimer's disease include pharmaceutical compositions of a STAT4 inhibitor, alone or in combination with one or more additional therapeutic agents, in a mixture with a physiologically compatible carrier, which can be administered to a subject, for example, a human subject, for therapeutic treatment. The presently disclosed pharmaceutical compositions can be administered using a variety of methods known in the art depending on the subject and/or the severity of the Alzheimer's disease. In an aspect, the STAT-4 inhibitor is administered, for example, orally, intranasally, by inhalation or intravenously.

The presently disclosed subject matter also includes the use of a STAT4 inhibitor, in the manufacture of a medicament for treatment of Alzheimer's disease. Regardless of the route of administration selected, the STAT4 inhibitor pharmaceutical compositions are formulated into pharmaceutically acceptable dosage forms such as described below or by other conventional methods known to those of skill in the art.

Actual dosage levels of the STAT4 inhibitor can be varied to obtain an amount of the active ingredient that is effective to achieve the desired therapeutic response for a particular subject without being toxic to the subject. The selected dosage level will depend on a variety of factors including the route of administration, the time of administration, the rate of excretion, the duration of the treatment, other drugs used in combination with the STAT4 inhibitor, the age, sex, weight, condition, general health, and prior medical history of the patient being treated, and like factors well known in the medical arts. A physician having ordinary skill in the art can readily determine and prescribe the effective amount of a given STAT4 inhibitor-containing pharmaceutical composition.

The presently disclosed compositions of a STAT4 inhibitor can be assembled into kits or pharmaceutical systems for use in treating Alzheimer's disease. In some embodiments, the presently disclosed kits or pharmaceutical systems include a STAT4 inhibitor in unit dosage form. In further embodiments, the STAT4 inhibitor can be present together with a pharmaceutically acceptable solvent, carrier, excipient, or the like, as described herein. In some embodiments, the presently disclosed kits include one or more containers, including, but not limited to a vial, tube, ampule, bottle, and the like, containing the STAT4 inhibitor. The presently disclosed kits or pharmaceutical systems also can include associated instructions for using the STAT4 inhibitor containing compositions for treating of Alzheimer's disease.

### BRIEF DESCRIPTION OF THE DRAWINGS

Various embodiments of the present disclosure are described herein with reference to the drawings.

FIG. 1A-B. FIG. 1A shows the time course, and FIG. 1B the mean±SEM magnitude, of the expression of LTP at Schaffer collateral-CA1 synapses in hippocampal slices in vitro, following the application of 3 theta burst trains of high frequency stimulation (arrow, TBS), in N slices per group. Stat4floxLysMcre denotes mice where the STAT4 gene has been knocked out, resulting in a complete absence of STAT4, while STAT4flox- denotes the control mice expressing normal levels of STAT4.

FIG. 2A-B. Control and STAT4 knockout mice challenged by prolonged feeding on a high-fat diet. The wildtype background control mice at 24 weeks of age exhibited robust LTP of approximately 1.6 times the pre-TBS baseline responses that lasted without reduction for at least 90 minutes recording time. When control mice (STAT4flox-) of the same background were fed a high-fat diet throughout the period from 8-24 weeks of age, the magnitude of LTP was statistically significantly reduced, indicating that this high-fat diet had markedly impaired the long-term, activity-dependent synaptic plasticity necessary for normal cognition, learning and memory. In contrast to the effects of high-fat diet in normal mice, mice lacking any STAT4 expression (Stat4floxLysMcre) did not exhibit impairments in expression of LTP. LTP in the absence of detectable STAT4 was significantly larger than in control mice fed either a normal or high-fat diet (exact P values shown in FIG. 2B, N=number of slices in each group).

FIG. 3A-B. Input/Output Relation for Baseline Synaptic Transmission in STAT4 KO v Control Mice at 3 weeks (FIG. 3A) or 24 weeks (FIG. 3B)

FIG. 4A-B. Presynaptic Stimulus-Evoked Paired-Pulse Profiles in STAT4 KO v Control Mice at 3 weeks (FIG. 4A) or 24 weeks (FIG. 4B).

### DETAILED DESCRIPTION

The presently disclosed subject matter provides methods for treating, delaying the onset, alleviating a symptom, and/or deterring the progression of Alzheimer's disease through administration of a STAT4 inhibitor in a therapeutically effective amount to a subject having, or at risk of developing Alzheimer's disease. In an embodiment, the subject to be treated may be one having a progressive neurodegenerative disorder that is suspected of having an inflammatory component. In a non-limiting embodiment, the subject to be treated may be one suffering from obesity, one having insulin resistance, cardiovascular disease such as atherosclerosis, and/or diabetes. While the disclosure below is directed to treatment of Alzheimer's disease, the disclosure applies equally as well to treatment or prevention of other neurodegenerative diseases that exhibit chronic, progressive neurological deficits, including but not limited to diabetes, Parkinson's Disease, Huntington's Disease, Lewy Body Disease, Vascular Disease, Frontotemperal Dementia and Chronic Traumatic Encephalopathy resulting from traumatic brain injury. Indeed, any neurological disease that involves central nervous system inflammation may be treated by drugs that target the STAT4 transcription factor activated inflammatory pathway. While not being bound to any theory, it is believed that STAT4 expression plays an important role in inflammation within neuronal tissue that, in turn, leads to development of progressive neurological deficits associated with diseases such as Alzheimer's disease. This is supported by the observed full protection of brain neuronal function in diabetic mice containing a STAT4 deletion specifically in immune cells as described below. Accordingly, administration of a STAT4 inhibitor can be used to reduce the symptoms and severity associated with neurodegenerative diseases such as Alzheimer's disease. The STAT4 inhibitor serves in this capacity by inhibiting STAT4 activity.

The administration of such STAT4 inhibitors are intended to reduce STAT4 mediated inflammation within the nervous system which, as demonstrated herein, has been found to be associated with development of Alzheimer's Disease. Targeted cells within the nervous system include, but are not limited to neurons, multiple glial cell types, vascular epithelial cells of the nervous system and those cells that contribute to inflammation within the nervous system. In one embodiment, the present disclosure provides a method of treating, preventing the progression or delaying the onset of Alzheimer's disease in a subject, the method comprising administering to the subject a therapeutically effective amount of at least one STAT4 inhibitor. In an embodiment, the subject to be treated may be one having a progressive neurodegenerative disorder that is suspected of having an inflammatory component. In a non-limiting embodiment, the subject to be treated may be one suffering from obesity, one having insulin resistance, cardiovascular disease such as atherosclerosis, and/or diabetes. “Delaying the onset of” a disease, e.g., Alzheimer's disease, or a symptom thereof means to defer, hinder, slow, retard, stabilize, and/or postpone development of the disease, or a symptom associated with, or resulting from the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of a disease, e.g., Alzheimer's disease, is a method that reduces probability of disease development in a given time frame and/or reduces extent of the disease in a given time frame, when compared to not using the method. Such comparisons are typically based on clinical studies, using a statistically significant number of subjects.

In one embodiment, the present disclosure provides a method of reducing the decline in functional or cognitive capacity in a subject diagnosed with early or mild to moderate Alzheimer's disease comprising administering to the subject suffering from early or mild to moderate Alzheimer's disease a therapeutically effective amount of at least one STAT4 inhibitor in an amount effective to slow the decline in functional or cognitive capacity in the subject.

In some embodiments, the decline in cognitive capacity may be assessed by determining the patient's score before and after administration of the antibody using a 12-item Alzheimer's disease Assessment Scale-Cognition (ADAS-Cog12), 13-item Alzheimer's disease Assessment Scale-Cognition (ADAS-Cog13), or 14-item Alzheimer's disease Assessment Scale-Cognition (ADAS-Cog14) test, optionally wherein the reduction in cognitive decline as measured by ADAS-Cog is at least 30%, at least 35%, at least 40%, or at least 45% relative to placebo.

As used herein, in general, a “therapeutically effective amount” of a STAT4 inhibitor refers to the amount of the agent necessary to elicit the desired biological response. In a specific embodiment, an effective amount is an amount sufficient for prevention or treatment of Alzheimer's disease and associated symptoms.

The effective amount of an agent may vary depending on such factors as the desired biological endpoint, the composition of the pharmaceutical composition, the target tissue or cell, the health of the subject to be treated and the like. In some embodiments, the term “therapeutic effective amount” refers to an amount sufficient to reduce or ameliorate the severity, duration, progression, or onset of symptoms associated with Alzheimer's disease.

A “subject” can include a human subject for medical purposes, such as for the treatment of Alzheimer's disease or the prophylactic treatment for preventing the onset of Alzheimer's disease, or an animal subject for medical, veterinary purposes, or developmental purposes. Further, a “subject” can include a patient afflicted with or suspected of being afflicted with Alzheimer's disease. Thus, the terms “subject” and “patient” are used interchangeably herein. In one embodiment, the presently disclosed subject matter relates to a method of treating or preventing Alzheimer's disease in a subject in need thereof, the method including administration to the subject of a therapeutically effective amount of a STAT4 inhibitor.

In some embodiments, the subject has a genomic mutation resulting in a genetic predisposition for developing Alzheimer's disease. In some embodiments, the Alzheimer's disease is sporadic or non-hereditary Alzheimer's disease. In some embodiments, the Alzheimer's disease is familial or hereditary Alzheimer's disease. In some embodiments, the Alzheimer's disease is clinical, pre-clinical, or prodromal Alzheimer's disease. In some embodiments, the subject has familial, and/or sporadic, and/or idiopathic Alzheimer's disease. In some embodiments, the subject has amyloid deposits. In some embodiments, the subject's brain has amyloid-beta amyloid deposits. In some embodiments, the subject is at risk of developing Alzheimer's disease. In some embodiments, the subject is selected from the group consisting of subjects with mild cognitive impairment, subjects with genotypes known to be associated with Alzheimer's disease, subjects with Trisomy 21, and subjects with surrogate markers indicating risk for Alzheimer's disease. In some embodiments, the subjects with genotypes known to be associated with Alzheimer's disease comprise subjects with the ApoE4 genotype.

As used herein, “Alzheimer's disease” means that the subject has symptoms associated with Alzheimer's disease. Such Alzheimer's disease symptoms include, for example, cognitive impairment, memory loss, depression, anxiety, dementia, irritability, confusion, mood swings, aggressive and/or apathetic behavior.

As used herein, the term “inhibit” or “inhibits” means to decrease, suppress, attenuate, diminish, arrest, or stabilize the development or progression of Alzheimer's disease by at least 10%, 20%, 40%, 40%, 50%, 60%, 70%, 80%, 90%, 95%, 98%, 99%, or even 100% compared to an untreated control subject. By the term “decrease” is meant to inhibit, suppress, attenuate, diminish, arrest, or stabilize a symptom of Alzheimer's disease. It should be appreciated that treating a disease, disorder or condition does not require that the disease, disorder, condition, or symptoms associated therewith be completely eliminated.

As used herein, the terms “treat,” treating,” “treatment,” and the like, are meant to decrease, suppress, attenuate, diminish, arrest, the underlying cause of Alzheimer's disease or to stabilize the development or progression of Alzheimer's disease and/or symptoms associated therewith. The terms “treat,” “treating,” “treatment,” and the like, as used herein can refer to curative therapy, prophylactic therapy, and preventative therapy. Accordingly, as used herein, “treating” means either slowing, stopping or reversing the progression of Alzheimer's disease, including reversing the progression to the point of eliminating the symptoms of Alzheimer's disease.

The treatment, administration, or therapy can be continuous or intermittent. Continuous treatment, administration, or therapy refers to treatment on at least a daily basis without interruption in treatment by one or more days. Intermittent treatment or administration, or treatment or administration in an intermittent fashion, refers to treatment that is not continuous, but rather cyclic in nature. Treatment according to the presently disclosed methods can result in complete relief or cure from Alzheimer's disease or partial amelioration of one or more symptoms of Alzheimer's disease and can be temporary or permanent.

As used herein, the terms “prevent,” “preventing,” “prevention,” “prophylactic treatment” and the like refer to reducing the probability of developing symptoms of Alzheimer's disease in a subject, who does not have, but is at risk of or susceptible to developing Alzheimer's disease. Thus, in some embodiments, a STAT4 inhibitor can be administered prophylactically to prevent the onset of Alzheimer's disease or to prevent the recurrence of Alzheimer's disease in a subject.

In certain embodiments, the presently disclosed subject matter also includes combination therapies. Additional therapeutic agents, which are normally administered to treat or prevent Alzheimer's disease, may be administered in combination with a STAT4 inhibitor as disclosed herein. For example, the STAT4 inhibitor may optionally be administered in conjunction with other compounds (e.g., therapeutic agents) or treatments useful in treating Alzheimer's disease. These additional agents may be administered separately, as part of a multiple dosage regimen, from the composition comprising a STAT4 inhibitor as disclosed herein. Alternatively, these agents may be part of a single dosage form, mixed together with a STAT4 inhibitor, in a single composition.

By “in combination with” is meant the administration of a STAT4 inhibitor, with one or more therapeutic agents either simultaneously, sequentially, or a combination thereof. Therefore, a subject can be administered a combination of a STAT4 inhibitor and one or more therapeutic agents at the same time (i.e., simultaneously) or at different times (i.e., sequentially, in either order, on the same day or on different days), so long as the effect of the combination of both agents is achieved in the subject. Where the STAT4 inhibitor and one or more therapeutic agents are administered simultaneously, they can be administered to the subject as separate pharmaceutical compositions, each containing either a STAT4 inhibitor or one or more therapeutic agents or be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times. In such combination therapies, the therapeutic effect of the first administered agent is not diminished by the sequential, simultaneous or separate administration of the subsequent agent(s).

Pharmaceutical compositions and formulations for use in treatment of Alzheimer's disease include pharmaceutical compositions of a STAT4 inhibitor, alone or in combination with one or more additional therapeutic agents, in a mixture with a physiologically compatible carrier, which can be administered to a subject, for example, a human subject, for therapeutic or prophylactic treatment. Such STAT4 inhibitors include small molecules that function to inhibit the activity of STAT4, including for example, those that inhibit the phosphorylation of serine and tyrosine residues said phosphorylation being demonstrated to activate STAT4 activity. Accordingly, inhibitors of JAK and other kinases known to phosphorylate STAT4 may be administered to inhibit the activity of STAT4. Inhibitors may also include those that prevent or reduce the dimerization of STAT4 or those that reduce or interfere with IL-12 signaling. Inhibitors include, for example, berbamine, STAT4 antisense oligonucleotides or oligodeoxynucleotide decoys to name a few.

In a specific embodiment, pharmaceutical compositions are provided comprising nucleic acid molecules designed to target STAT4 mRNA and inhibit, silence or attenuated the expression of that RNA for use in methods for prevention or treatment of Alzheimer's disease. The terms “inhibit,” “silencing,” and “attenuating” can refer to a measurable reduction in expression of a target mRNA (or the corresponding polypeptide or protein) as compared with the expression of the target mRNA (or the corresponding polypeptide or protein) in the absence of an interfering RNA molecule. The reduction in expression of the target mRNA (or the corresponding polypeptide or protein) is commonly referred to as “knock-down” and is reported relative to levels present following administration or expression of a non-targeting control RNA.

The term “antisense” is used in reference to RNA sequences which are complementary to a specific RNA sequence (e.g., mRNA). Antisense RNA may be produced by any method, including synthesis by splicing the gene(s) of interest in a reverse orientation to a viral promoter which permits the synthesis of a coding strand. Once introduced into a cell, this transcribed strand combines with natural mRNA produced by the cell to form duplexes. These duplexes then block either the further transcription of the mRNA or its translation. The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

The terms “siRNA” refers to either small interfering RNA, short interfering RNA, or silencing RNA. Generally, siRNA comprises a class of double-stranded RNA molecules, approximately 20-25 nucleotides in length. Most notably, siRNA is involved in RNA interference (RNAi) pathways and/or RNAi-related pathways, wherein the compounds interfere with gene expression.

The term “shRNA” refers to any small hairpin RNA or short hairpin RNA. Although it is not necessary to understand the mechanism of action, it is believed that any sequence of RNA that makes a tight hairpin turn can be used to silence gene expression via RNA interference. Typically, shRNA uses a vector introduced into a cell genome and is constitutively expressed by a compatible promoter. The shRNA hairpin structure may also be cleaved into siRNA, which may then become bound to the RNA-induced silencing complex (RISC). This complex binds to and cleaves mRNAs which match the siRNA that is bound to it.

The term “microRNA” or “miRNA”, refers to any single-stranded RNA molecules of approximately 21-23 nucleotides in length, which regulate gene expression. miRNAs may be encoded by genes from whose DNA they are transcribed but miRNAs are not translated into protein (i.e. they are non-coding RNAs). Each primary transcript (a pri-miRNA) is processed into a short stem-loop structure called a pre-miRNA and finally into a functional miRNA. Mature miRNA molecules are partially complementary to one or more messenger RNA (mRNA) molecules, and their main function is to down-regulate gene expression.

Methods for design and expression of such nucleic acids, e.g., antisense, miRNA, siRNA and shRNA, are well known to those of skill in the art. Such inhibitory nucleic acids may be designed based on the publicly available sequence of the STAT4 gene.

In an embodiment, antibody molecules that bind to STAT4 may also be used inhibit the activity of STAT4 in Alzheimer's disease subjects. “Antibody molecule” as used herein is intended to include intact antibodies, such as polyclonal antibodies or monoclonal antibodies (mAbs), as well as proteolytic fragments thereof such as the Fab or F(ab′)2 fragments, chimeric antibodies, nanobodies, recombinant and engineered antibodies, single-chain antibodies and fragments thereof, as well as other molecules having at least one STAT4 antigen-binding site. Antibody molecules that also inhibit JAK and other kinases known to phosphorylate STAT4 may be administered to inhibit the activity of STAT4. Inhibitors also include antibodies may function to prevent or reduce the dimerization of STAT4 or those that reduce or interfere with IL-12 signaling.

Also provided is a nanoparticle comprising a STAT4 inhibitor for use in treatment of Alzheimer's disease. Such nanoparticles can be natural or synthetic and may be incorporated into a vaccine composition. They can be created from biological molecules or from non-biological molecules. In some cases, the protein complex is crosslinked to a polymer or lipid on nanoparticle surface. In embodiments, the protein complex is adsorbed onto the nanoparticle surface. In some embodiments, the protein complex is adsorbed onto the nanoparticle surface and then crosslinked to the nanoparticle surface. In some embodiments, the protein complex is encapsulated into the nanoparticle.

In particular embodiments, the nanoparticle is formed from a biocompatible polymer. Examples of biocompatible polymers include polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, or combinations thereof. In some cases, the nanoparticle is formed from a polyethylene glycol (PEG), poly(lactide-co-glycolide) (PLGA), polyglycolic acid, poly-beta-hydroxybutyrate, polyacrylic acid ester, or a combination thereof. In a specific embodiment the nanoparticle is a nanoliposome. Such nanoliposomes may be composed of phospholipids such as 1,2-distearoyl-sn-glycero-3-phosphocholine (DSPC), 1,2-dipalmitoyl-sn-glycero-3-phosphocholine (DPPC), 1,2-dimyristoyl-sn-glycero-3-phosphocholine (DMPC), 1,2-dioleoyl-sn-glycero-3-phosphocholine (DOPC), 1,2-distearoyl-sn-glycero-3-phospho-(1′-rac-glycerol) (DSPG), 1,2-dipalmitoyl-sn-glycero-3-phospho-(1′-rac-glycerol) (DPPG), 1,2-dimyristoyl-sn-glycero-3-phospho-(1′-rac-glycerol) (DMPG), 1,2-dioleoyl-sn-glycero-3-phospho-(1′-rac-glycerol) (DOPG), dipalmitoyl phosphatidylserine (DPPS), distearoyl phosphatidylserine (DSPS), dipalmitoyl phosphatidylinositol (DPPI), distearoyl phos phatidylinositol (DSPI), dipalmitoyl phosphatidic acid (DPPA), distearoyl phosphatidic acid (OSPA), 1,2-diacyl trimethylammonium-propanes, (including but not limited to, dioleoyl (DOTAP), 1,2-dipalmitoyl-sn-glycero-3-phosphoethanolamine-N [methoxy(polyethylene glycol)-2000] (DPPE-PEG2000), 1,2-distearoyl-sn-glycero-3-phosphoethanolamine-N-[methoxy(polyethylene glycol)-1000] (DSPE-PEG2000), and cholesterol.

In some embodiments, the STAT4 inhibitor is coated on the nanoparticle using a crosslinking agent. In some embodiments, the STAT4 inhibitor is adsorbed onto the nanoparticle surface. In some embodiments, the STAT4 inhibitor is adsorbed onto the nanoparticle surface followed by covalent crosslinking of the STAT4 inhibitor to the nanoparticle surface using a crosslinking agent.

Crosslinking agents suitable for crosslinking the STAT4 inhibitor to produce the nanoparticle, or to coat SC-membrane protein on the nanoparticle are known in the art, and include those selected from the group consisting of formaldehyde, formaldehyde derivatives, formalin, glutaraldehyde, glutaraldehyde derivatives, a protein cross-linker, a nucleic acid cross-linker, a protein and nucleic acid cross-linker, primary amine reactive crosslinkers, sulfhydryl reactive crosslinkers, sulfydryl addition or disulfide reduction, carbohydrate reactive crosslinkers, carboxyl reactive crosslinkers, photoreactive crosslinkers, cleavable crosslinkers, AEDP, APG, BASED, BM(PEO)3, BM(PEO)4, BMB, BMDB, BMH, BMOE, BS3, BSOCOES, DFDNB, DMA, DMP, DMS, DPDPB, DSG, DSP, DSS, DST, DTBP, DTME, DTSSP, EGS, HBVS, sulfo-BSOCOES, Sulfo-DST, and Sulfo-EGS.

Pharmaceutical compositions and formulations for use in treatment of Alzheimer's disease include pharmaceutical compositions comprising an effective amount of a STAT4 inhibitor and a physiologically compatible carrier, which can be administered to a subject, for example, a human subject, for therapeutic or prophylactic treatment of Alzheimer's disease. As used herein, “physiologically compatible carrier” refers to a physiologically acceptable diluent including, but not limited to water, phosphate buffered saline, or saline, and, in some embodiments, can include an adjuvant. Acceptable carriers, excipients, or stabilizers are nontoxic to recipients at the dosages and concentrations employed, and can include buffers such as phosphate, citrate, and other organic acids; antioxidants including ascorbic acid, BHA, and BHT; low molecular weight (less than about 10 residues) polypeptides; proteins, such as serum albumin, gelatin or immunoglobulins; hydrophilic polymers, such as polyvinylpyrrolidone, amino acids such as glycine, glutamine, asparagine, arginine, or lysine; monosaccharides, disaccharides, and other carbohydrates including glucose, mannose, or dextrans; chelating agents such as EDTA; sugar alcohols such as mannitol or sorbitol; salt-forming counter-ions such as sodium; and/or nonionic surfactants such as Tween, Pluronics, or PEG. Adjuvants suitable for use with the presently disclosed compositions include adjuvants known in the art including, but not limited to, incomplete Freund's adjuvant, aluminum phosphate, aluminum hydroxide, and alum.

As used herein, “physiologically compatible carrier” includes any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference).

The presently disclosed pharmaceutical compositions can be administered using a variety of methods known in the art. More particularly, as described herein, the STAT4 inhibitor can be administered to a subject for treatment of Alzheimer's disease by any suitable route of administration, including orally, nasally, transmucosally, parenterally, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections, intracisternally, topically, as by powders, ointments, including buccally and sublingually, transdermally, through an inhalation spray, or other modes of delivery known in the art.

In one embodiment, the present disclosure provides a method for treating the neuronal or brain tissue of a subject comprising administering a STAT4 composition that is permeable to the blood-brain barrier (BBB). Targeted cells of the nervous system include but are not limited to neurons, glial cells, vascular epithelial cells and/or inflammatory cells of the nervous system. Means for delivery of STAT4 inhibitors across the blood brain barrier include, for example, the use of viral vectors, nanoparticles, exosomes, brain permeability enhancers or by intravenous injection. Delivery across the blood brain barrier may also be achieved through the permeability of the blood-brain barrier under disease conditions or by bypass of the blood-brain barrier through nasal administration. For example, inflammation of cerebral blood vessels can lead to a “leaky” blood brain barrier thereby facilitating the transfer of STAT4 inhibitors into cells of the nervous system.

Parenteral administration, in particular intravenous injection, may be used for administering of STAT4 inhibitors. Aqueous injection suspensions may contain compounds which increase the viscosity of the suspension. Optionally, the suspension may also contain suitable stabilizers or agents which increase the solubility of the compounds to allow for the preparation of highly concentrated solutions. Parenteral compositions include those designed for administration by injection, e.g. subcutaneous, intradermal, intra-lesional, intravenous, intra-arterial, intramuscular, intrathecal or intraperitoneal injection.

In one embodiment, the present disclosure provides a method for treating a subject comprising forming an aerosol of a dispersion of particles, wherein the particles comprise a STAT4 inhibitor and an additive that enhances absorption of the drug into tissue of the respiratory system and administering the aerosol to the respiratory system of the subject. Pulmonary drug delivery of the STAT4 inhibitor is accomplished by inhalation of an aerosol through the mouth and throat. In an embodiment, the pharmaceutical compositions may be formulated for pulmonary delivery. Optimized formulations for such delivery may include addition of permeability enhancers (mucoadhesives, nanoparticles, and the like) as well as combined use with a pulmonary drug delivery device (for example, one that provides controlled particle dispersion with particles aerosolized to target the upper nasal cavity).

Pharmacologically active amounts of the drug substance, i.e., STAT4 inhibitors, are thereby delivered to the circulation or directly to a site of action. The present disclosure relates to a dosage form (for example a nasal spray, a nasal gel, a nasal ointment, inhalation solutions, inhalation suspensions, inhalation sprays, dry powder or an aerosol) which is specifically designed or adapted for administration of a drug substance to the nasal structures. Such dosage forms may be used as a means for bypass of the blood brain barrier for delivery to brain tissue. Hence in one aspect the disclosure relates to the STAT4 inhibitors disclosed herein for use in a therapeutic method, wherein the drug substance is administered intranasally. In an embodiment, administration is to the upper nasal cavity. The pharmaceutical compositions may be delivered using an inhalator.

A pharmaceutical composition may be prepared, packaged, or sold in a formulation suitable for pulmonary administration via the buccal cavity. Such a formulation may include dry particles that contain the active ingredient and have a diameter in the range from about 0.5 to about 7 nanometers, and in certain embodiments from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device including the active ingredient dissolved or suspended in a low-boiling propellant in a sealed container.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further contain additional ingredients such as a liquid non-ionic or solid anionic surfactant or a solid diluent (in certain embodiments having a particle size of the same order as particles comprising the active ingredient).

Pharmaceutical compositions formulated for delivery may also provide the STAT4 inhibitor in the form of droplets of a solution or suspension. Such formulations may be prepared, packaged, or sold as aqueous or dilute alcoholic solutions or suspensions, optionally sterile, including the active ingredient, and may conveniently be administered using any nebulization or atomization device. Such formulations may further include one or more additional ingredients including, but not limited to, a flavoring agent such as saccharin sodium, a volatile oil, a buffering agent, a surface active agent, or a preservative such as methylhydroxybenzoate. The droplets provided by this route of administration in certain embodiments have an average diameter in the range from about 0.1 to about 200 nanometers.

In one aspect, the STAT4 inhibitor is administered orally. When a therapeutically effective amount of the composition(s) is administered orally, it may be in the form of a solid or liquid preparation such as capsules, pills, tablets, lozenges, melts, powders, suspensions, solutions, elixirs or emulsions. Solid unit dosage forms can be capsules of the ordinary gelatin type containing, for example, surfactants, lubricants, and inert fillers such as lactose, sucrose, and cornstarch, or the dosage forms can be sustained release preparations. The pharmaceutical composition(s) may contain a solid carrier, such as a gelatin or an adjuvant. The tablet, capsule, and powder may contain from about 0.05 to about 95% of the STAT4 inhibitor compound by dry weight. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition(s) may further contain physiological saline solution, dextrose or other saccharide solution, or glycols such as ethylene glycol, propylene glycol, or polyethylene glycol.

In another embodiment, the composition(s) of the present disclosure can be tableted with conventional tablet bases such as lactose, sucrose, and cornstarch in combination with binders, such as acacia, cornstarch, or gelatin, disintegrating agents such as potato starch or alginic acid, and a lubricant such as stearic acid or magnesium stearate. Liquid preparations are prepared by dissolving the composition(s) in an aqueous or non-aqueous pharmaceutically acceptable solvent which may also contain suspending agents, sweetening agents, flavoring agents, and preservative agents as are known in the art.

In some embodiments, the presently disclosed pharmaceutical compositions can be administered by rechargeable or biodegradable devices. For example, a variety of slow-release polymeric devices have been developed and tested in vivo for the controlled delivery of drugs. Suitable examples of sustained release preparations include semipermeable polymer matrices in the form of shaped articles, e.g., films or microcapsules. Sustained release matrices include polyesters, hydrogels, polylactides (U.S. Pat. No. 3,773,919; EP 58,481), copolymers of L-glutamic acid and gamma ethyl-L-glutamate (Sidman et al., Biopolymers 22:547, 1983), poly (2-hydroxyethyl-methacrylate) (Langer et al., J. Biomed. Mater. Res. 15:167, 1981; Langer, Chem. Tech. 12:98, 1982), ethylene vinyl acetate (Langer et al., Id), or poly-D-(−)-3-hydroxybutyric acid (EP 133,988A). Sustained release compositions also include liposomally entrapped a STAT4 inhibitor, which can be prepared by methods known per se (Epstein et al., Proc. Natl. Acad. Sci. U.S.A. 82:3688, 1985; Hwang et al., Proc. Natl. Acad. Sci. U.S.A. 77:4030, 1980; U.S. Pat. Nos. 4,485,045 and 4,544,545; and EP 102,324A). Ordinarily, the liposomes are of the small (about 200-800 Angstroms) unilamelar type in which the lipid content is greater than about 30 mol % cholesterol, the selected proportion being adjusted for the optimal therapy. Such materials can include an implant, for example, for sustained release of the STAT4 inhibitor.

The presently disclosed subject matter also includes the use of a STAT4 inhibitor, in the manufacture of a medicament for treatment of Alzheimer's disease. Regardless of the route of administration selected, the STAT4 inhibitor pharmaceutical compositions are formulated into pharmaceutically acceptable dosage forms such as described herein or by other conventional methods known to those of skill in the art.

Actual dosage levels of the STAT4 inhibitor can be varied to obtain an amount of the active ingredient that is effective to achieve the desired therapeutic response for a particular subject, composition, route of administration, and disease, disorder, or condition without being toxic. The selected dosage level will depend on a variety of factors including the route of administration, the time of administration, the rate of excretion, the duration of the treatment, other drugs used in combination with the STAT4 inhibitor, the age, sex, weight, condition, general health and prior medical history of the patient being treated, and like factors well known in the medical arts.

A physician having ordinary skill in the art can readily determine and prescribe the effective amount of a STAT4 inhibitor-containing pharmaceutical composition required. For example, the physician could start doses of the STAT4 inhibitor lower than that required to achieve the desired therapeutic effect and gradually increase the dosage until the desired effect is achieved. Accordingly, the dosage range for administration will be adjusted by the physician, as necessary. It will be appreciated that an amount of a STAT4 inhibitor required for achieving the desired biological response, e.g., treatment or prevention of Alzheimer's disease, may be different from the amount of a STAT4 inhibitor effective for another purpose.

In general, a suitable daily dose of a STAT4 inhibitor will be that amount of the drug that is the lowest dose effective to produce a therapeutic effect. Such an effective dose will generally depend upon the factors described above. Effective dosages may be determined based generally on the weight of the subject to be treated. If desired, the effective daily dose of the STAT4 inhibitor can be administered as one, two, three, four, five, six or more sub-doses administered separately at appropriate intervals throughout the day, optionally, in unit dosage forms.

In an embodiment, pharmaceutical compositions containing a STAT4 inhibitor are provided for use in a method of treating or ameliorating the symptoms in a subject having Alzheimer's disease.

The presently disclosed compositions of a STAT4 inhibitor can be assembled into kits or pharmaceutical systems for use in treating or preventing Alzheimer's disease. In some embodiments, the presently disclosed kits or pharmaceutical systems include a STAT4 inhibitor in unit dosage form. In further embodiments, the STAT4 inhibitor can be present together with a pharmaceutically acceptable solvent, carrier, excipient, or the like, as described herein.

In some embodiments, the presently disclosed kits include one or more containers, including, but not limited to a vial, tube, ampule, bottle, and the like, for containing the STAT4 inhibitor. The one or more containers also can be carried within a suitable carrier, such as a box, carton, tube, or the like. Such containers can be made of plastic, glass, laminated paper, metal foil, or other materials suitable for holding medicaments.

The presently disclosed kits or pharmaceutical systems also can include associated instructions for using the STAT4 inhibitor containing compositions for treating Alzheimer's disease. In some embodiments, the instructions include one or more of the following: a description of pharmaceutical composition containing a STAT4 inhibitor; a dosage schedule and administration for treating or preventing Alzheimer's disease; precautions; warnings; indications; counter-indications; overdosage information; adverse reactions; animal pharmacology; clinical studies; and references. The instructions can be printed directly on a container (when present), as a label applied to the container, as a separate sheet, pamphlet, card, or folder supplied in or with the container.

### Example

The present study examined the effects of elimination of STAT4 signaling in transgenic mice lacking STAT4 on the expression of long-term potentiation (LTP) of synaptic transmission, and learning and memory, in mice fed normal versus high-fat diets. Electrophysiological recordings from Schaffer collateral-CA1 synapses in hippocampal slices from 3 or 24 week old LDLr−/−STAT4flox/floxLysMcre (LDLr−/− crossed to STAT4flox-Lyz2<tml(cre)Ifo>/J) mice showed no differences in baseline synaptic transmission, paired-pulse facilitation (PPF, a measure of presynaptic function), or LTP compared to age-matched LDLr−/−STAT4flox/flox wildtype littermates. Feeding mice a high-fat diet from 8-24 weeks of age resulted in marked, significant impairments in LTP compared to age-matched mice fed a normal diet, without differences in basal transmission or PPF. In contrast to wildtype LDLr−/−STAT4flox/flox mice, LDLr−/−STAT4 flox/flox LysMcre mice receiving the same high-fat diet were completely protected from the high-fat diet-associated reduction in LTP. These findings were correlated with protection of cognitive function in behavioral learning and memory tasks from the effects of the same high-fat diet. Taken together, the data support an essential role for the inflammatory transcription factor STAT4 in the sensitivity of the central nervous system to high-fat diet-induced impairments in synaptic plasticity and cognitive function, and provide STAT4 pathways as a new target for interventions to treat neurodegenerative diseases such as Alzheimer's, where chronic neuroinflammation is a critical disease mediator.

The following experimentation demonstrates a connection between STAT4 and long-term, activity-dependent synaptic plasticity that is essential for human cognition and memory. It is known that type 2 diabetes, vascular disease, and atherosclerosis, are associated with a higher risk of developing Alzheimer's disease, and that Alzheimer's disease is linked to premature impairments in the magnitude and stability of long-term synaptic plasticity underlying long-term memory formation. Demonstrated herein is that STAT4 plays a key regulatory role in the expression of long-term, activity-dependent synaptic plasticity. Since this plasticity is critical to the storage and retrieval of long-term memories, this offers a completely new molecular target and mechanisms for treatment of Alzheimer's disease that may be utilized to slow, or even halt, the progression of this and other neurodegenerative diseases that exhibit chronic, progressive neurological deficits, such as Diabetes, Parkinson's disease, Huntington's disease, Lewy Body disease, and Chronic Traumatic Encephalopathy. Indeed, any neurological disease that involves central nervous system inflammation has the potential to be treated by drugs that target the STAT4 transcription factor activated inflammatory pathway.

The following observations have been made. First, feeding normal mice a high-fat diet for 16 weeks, from 8-24 weeks of age (20-34 years of age human equivalent), statistically significantly reduces long-term, activity-dependent synaptic potentiation (LTP), compared to mice of the same age fed a normal diet. Second, the absence of the gene that codes for the inflammatory transcription factor STAT4, completely prevents the decline in long-term, activity-dependent synaptic potentiation (LTP) produced by feeding a high-fat diet to mice. Third, neither the high-fat diet, nor absence of the gene for STAT4, alters basal synaptic transmission strength. Finally, neither the high-fat diet, nor absence of the gene for STAT4, alters presynaptic release of glutamate, as assessed by the response of neurons to paired-pulse stimulation that is known to elicit presynaptic potentiation of glutamate release.

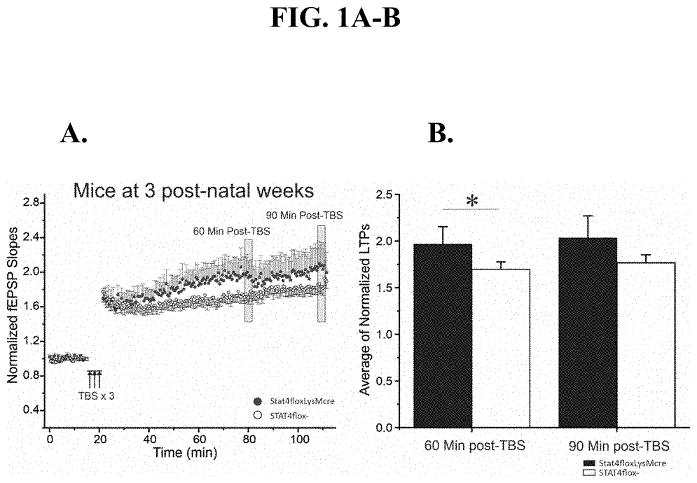

FIG. 1A shows the time course, and FIG. 1B the mean±SEM magnitude, of the expression of LTP at Schaffer collateral-CA1 synapses in hippocampal slices in vitro, following the application of 3 theta burst trains of high frequency stimulation (arrow, TB S). Stat4floxLysMcre denotes mice where the STAT4 gene has been knocked out, resulting in a complete absence of STAT4, while STAT4flox- denotes the control mice expressing normal levels of STAT4. At three weeks of age, LTP was significantly larger 60 minutes post-theta burst stimulation (*P<0.05, Student's t-test), but there is no difference in the magnitude or duration of LTP by 90 minutes post-theta burst stimulation, in the absence or presence of normal STAT4 expression levels. (60 min: Stat4floxLysMcre N=23 slices; STAT4flox- N=44 slices; 90 min: Stat4floxLysMcre N=20 slices; STAT4flox- N=36 slices).

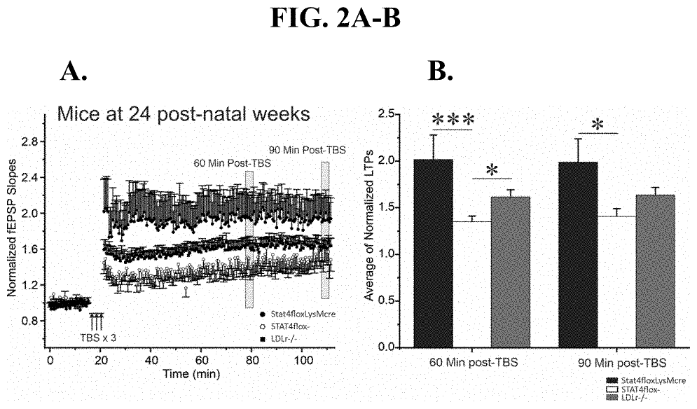

In contrast to young 3 week old mice fed a normal diet, when control and STAT4 knockout mice were challenged by prolonged feeding on a high-fat diet, their responses were very different. FIG. 2A-B illustrates these findings. The wildtype background control mice at 24 weeks of age, data shown in filled squares and grey bars, exhibited robust LTP of approximately 1.6 times the pre-TBS baseline responses that lasted without reduction for at least 90 minutes recording time. When control mice (STAT4flox-, open circles and open bars) of the same background were fed a high-fat diet throughout the period from 8-24 weeks of age, the magnitude of LTP was statistically significantly reduced (*P<0.05, Student's t-test), indicating that this high-fat diet had markedly impaired the long-term, activity-dependent synaptic plasticity necessary for normal cognition, learning and memory.

In contrast to the effects of high-fat diet in normal mice, mice lacking any STAT4 expression (Stat4floxLysMcre, filled circles and black bars) did not exhibit impairments in expression of LTP. In fact, LTP in the absence of detectable STAT4 was significantly larger than in control mice fed either a normal or high-fat diet (*P<0.05, ***P<0.001; Stat4floxLysMcre N=12 slices, STAT4flox- N=16, LDLr−/− N=28). These are the key findings in the discovery that STAT4 normally contributes to the chronic effects of a high-fat diet that impair long-term synaptic plasticity and supports the hypothesis that this inflammatory transcription factor that is a key contributor to the increased risk of Alzheimer's disease in type 2 diabetes. Previous studies revealed that STAT4 plays a critical role in inflammation in the cardiovascular system, leading to the proposal that it also acts via vascular inflammation it the brain to accelerate the progression of the neuroinflammatory disease known as Alzheimer's disease. This leads to the conclusion that other neurodegeneration diseases with inflammatory components that speed their progression will also be slowed or prevented entirely by agents that block the functions of STAT4.

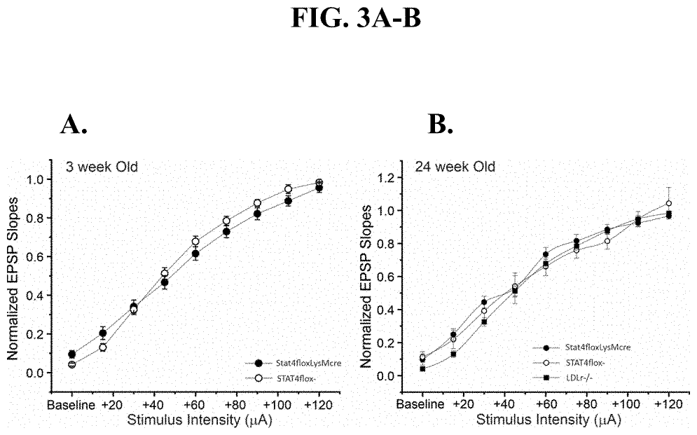

FIG. 3A-B shows the input/output relationships of normalized EPSP amplitude (y axes) to stimulus intensity (x axes) at 3 (N=22 Control slices, 21 STAT4 Knockout) and 24 (N=15 Control slices, 15 STAT4 Knockout, 23 Control Low Fat Diet) weeks of age when consuming either normal or high-fat diets. STAT4 knockout (filled circles) and control mice (open circles) on either normal (filled squares) or high-fat (filled and open circles) diets showed synaptic strength profiles that were not significantly different from each other, indicating that baseline synaptic transmission was not significantly altered by either the absence of STAT4, or the consumption of a high-fat diet from 8-24 weeks of age in either normal control or STAT4 knockout mice.

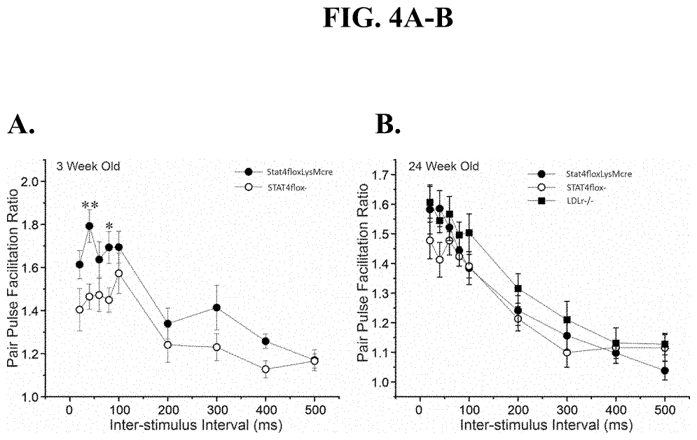

FIG. 4A-B shows the paired-pulse profiles of the ratios of second to first fEPSP slope magnitude as a function of inter-stimulus interval between the first and second stimulus, in milliseconds at 3 (N=12 Control slices, 15 STAT4 Knockout) and 24 (N=16 Control slices, 10 Knockout, 14 Control Low Fat Diet) weeks of age when consuming either normal or high-fat diets. STAT4 KO and control mice on either normal (filled squares) or high-fat (filled and open circles) diets showed paired-pulse profiles that were not significantly different from each other, except for the 20 msec inter-stimulus interval at 3 weeks of age (*, P<0.05, Repeated Measures ANOVA and post-hoc t-test with Bonferroni correction), indicating that presynaptic function was not substantially he altered by either the absence of STAT4, or the consumption of a high-fat diet from 8-24 weeks of age, in either normal control or STAT4 knockout mice.

What is claimed:

1. A method of treating, or preventing, a neurodegenerative disorder in a subject in need thereof, the method comprising administration to the subject of a therapeutically effective dose of a STAT4 inhibitor.

2. The method of claim 1, wherein the neurodegenerative disorder is associated with inflammation in the brain tissue of the subject.

3. The method of claim 1, wherein the neurodegenerative disorder is selected from the group consisting of diabetes, Parkinson's Disease, Huntington's Disease, Lewy Body Disease, Vascular Disease, Frontotemperal Dementia, Chronic Traumatic Encephalopathy and Alzheimer's disease.

4. The method of claim 3, wherein the neurodegenerative disorder is Alzheimer's disease.

5. The method of claim 1, wherein the STAT4 inhibitor is an inhibitory nucleic acid.

6. The method of claim 1, wherein the STAT4 inhibitor is a small molecule.

7. The method of claim 1, wherein the STAT4 inhibitor is an antibody.

8. The method of claim 1, wherein the STAT4 inhibitor is an inhibitor of STAT4 phosphorylation.

9. The method of claim 1, wherein the STAT4 inhibitor is an inhibitor of STAT4 dimerization.

10. The method of claim 1, further comprising administration of a second Alzheimer's disease therapeutic agent.

11. A kit comprising: (i) one or more containers for containing a STAT4 inhibitor-containing compositions; and (ii) instructions for using the STAT4 inhibitor containing compositions for treatment of Alzheimer's disease.

12. The kit of claim 11, wherein the instructions include one or more of the following: (i) a description of pharmaceutical composition containing a STAT4 inhibitor; (ii) a dosage schedule; or (iii) instructions for administration of the pharmaceutical composition containing a STAT4 inhibitor for treatment of Alzheimer's disease.
